(19) United States
(12) Patent Application Publication
(10) Pub. No.: US 2017/0333655 A1
(43) Pub. Date: Nov. 23, 2017

(54) ENDOTRACHEAL TUBE PROBE

(71) Applicant: Alaaeldin Soliman, Holmdel, NJ (US)

(72) Inventor: Alaaeldin Soliman, Holmdel, NJ (US)

(21) Appl. No.: 15/157,148

(22) Filed: May 17, 2016

(51) Int. Cl. A61M 16/04; A61B 5/06

(52) U.S. Cl. CPC A61M 16/0488; A61B 5/062; A61M 2205/0272

(57) ABSTRACT

An endotracheal (ET) tube probe system is configured to identify a proper placement of an ET tube that is placed inside a human body. The system includes a longitudinal member having a first end and an oppositely positioned second end; a magnet positioned at the second end; at least one positional indicator arranged on the longitudinal member; and a mechanism having at least one metallic needle, wherein the longitudinal member is configured to be inserted into the ET tube that is placed in the human body, and wherein the at least one metallic needle is configured to align with the magnet when the mechanism is placed adjacent to and outside of the human body. The top of the at least one positional indicator may be adapted to align with a top of the ET tube to indicate a proper placement of the longitudinal member relative to the ET tube.

### BACKGROUND

### Technical Field

The embodiments herein generally relate to medical devices, and more particularly to endotracheal tubes.

### Description of the Related Art

Mechanical ventilation is provided to patients with respiratory failure, and is provided using an endotracheal (ET) tube that connects a ventilator machine to the patient's lungs. The ET tube is placed through the mouth or nose into the larynx and down to the trachea where it remains while intubation is needed, and in most emergency situations the ET tube is placed through the mouth. Most ET tubes are made of polyvinyl chloride (PVC), silicone rubber, latex rubber, or stainless steel, and are typically a standard length of approximately 30 cm. The length of the human trachea is approximately 10 cm. The best placement location of the ET tube is when the tip of the ET tube is in the middle of the trachea. Current medical practice uses the patient's lips as a measure at which the ET tube is advanced until the 22 cm mark to approximately predict that the ET tube tip is in the middle of the trachea. This is a very crude technique and still requires an X-ray to confirm the appropriate location of the tube tip and is subject to a large number of errors sometimes requiring re-placement of the ET tube and another X-ray to confirm proper positioning. During medical emergencies these inefficiencies can mean the difference between life and death. The lips are generally not an accurate location as a marker to determine how deep the ET tube should be inserted into the trachea because from the lips to the middle of the trachea (e.g., approximately 22 cm) there are several anatomical variations such as mouth size, teeth or no teeth, head size, neck size, and the location of the larynx in the neck varies from high to low from patient-to-patient. Therefore, it is desirable to develop a new and improved technique for proper ET placement in a patient.

### SUMMARY

In view of the foregoing, an embodiment herein provides an ET tube probe system configured to identify a proper placement of an ET tube that is placed inside a human body, the system comprising a longitudinal member comprising a first end and an oppositely positioned second end; a magnet positioned at the second end of the longitudinal member; at least one positional indicator arranged on the longitudinal member; and a mechanism comprising at least one metallic needle, wherein the longitudinal member is configured to be inserted into the ET tube that is placed in the human body, and wherein the at least one metallic needle is configured to align with the magnet when the mechanism is placed adjacent to and outside of the human body. The longitudinal member may comprise plastic. The at least one positional indicator may comprise markings on the longitudinal member. The longitudinal member may comprise a first width, and the at least one positional indicator may comprise a second width. The first width may be different than the second width. The magnet may be thicker than the longitudinal member. The top of the at least one positional indicator may be adapted to align with a top of the ET tube to indicate a proper placement of the longitudinal member relative to the ET tube. The longitudinal member is freely movable relative to the ET tube. The longitudinal member is removable from the ET tube. The at least one metallic needle may comprise a pair of metallic needles. The pair of metallic needles is configured to become horizontally positioned with respect to one another upon alignment with the magnet. The mechanism may comprise a space that separates the pair of metallic needles from each other. The space is configured to be positioned over the magnet upon proper placement of the ET tube in the human body, wherein the at least one metallic needle aligns with the magnet upon the proper placement.

Another embodiment provides a method of identifying a proper placement of an ET tube that is placed inside a human body, the method comprising placing an ET tube in the human body; providing a longitudinal member inside the ET tube, wherein the longitudinal member comprises a first end, an oppositely positioned second end, a magnet positioned at the second end, and at least one positional indicator arranged on the longitudinal member; inserting the longitudinal member such that a top of the at least one positional indicator aligns with a top of the ET tube; providing a mechanism comprising a pair of metallic needles; positioning the mechanism adjacent to and outside of the human body at a location proximate to the where the magnet is positioned inside the ET tube; and aligning the pair of metallic needles with the magnet. The method may further comprise moving both the longitudinal member and the ET tube until the pair of metallic needles are horizontally positioned with respect to one another. The moving of both the longitudinal member and the ET tube until the pair of metallic needles is horizontally positioned with respect to one another occurs simultaneously such that the longitudinal member and the ET tube move in unison. A top of the at least one positional indicator is adapted to align with a top of the ET tube to indicate a proper placement of the longitudinal member relative to the ET tube. The longitudinal member is freely movable relative to the ET tube. The longitudinal member is removable from the ET tube.

Another embodiment provides a method of identifying a proper placement of an ET tube that is placed inside a human body, the method comprising providing a mechanism comprising a pair of metallic needles; positioning the mechanism adjacent to and outside of the human body; providing an ET tube; providing a longitudinal member comprising a magnet and at least one positional indicator arranged on the longitudinal member; inserting the longitudinal member in the ET tube such that a top of the at least one positional indicator aligns with a top of the ET tube; inserting the ET tube comprising the longitudinal member inside the human body; and adjusting an insertion distance of the inserted ET tube based on an indication of the pair of metallic needles being aligned with the magnet.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a cross-sectional perspective view of a system for placing and locating an ET tube according to an embodiment herein;

FIG. 2 illustrates a cross-sectional plan view of the system of FIG. 1 according to an embodiment herein;

FIG. 3 illustrates another cross-sectional plan view of the system of FIG. 2 according to an embodiment herein;

FIG. 4 illustrates a perspective view of an ET tube probe inserted into a larynx and trachea according to an embodiment herein;

FIG. 5 illustrates a plan view of the ET tube probe inserted into a larynx and trachea according to an embodiment herein;

FIG. 6 illustrates a cross-sectional perspective view of the ET tube probe inserted into a larynx and trachea according to an embodiment herein;

FIG. 7 illustrates a magnified cross-sectional perspective view of the ET tube probe inserted into a larynx and trachea according to another embodiment herein;

FIG. 8 illustrates an enhanced magnified cross-sectional perspective view of the ET tube probe inserted into a larynx and trachea according to still another embodiment herein;

FIG. 9 illustrates a cross-sectional plan view of the ET tube probe inserted into a larynx and trachea according to an embodiment herein;

FIG. 10 is a flow diagram illustrating a method according to an embodiment herein; and

FIG. 11 is a flow diagram illustrating a method according to another embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

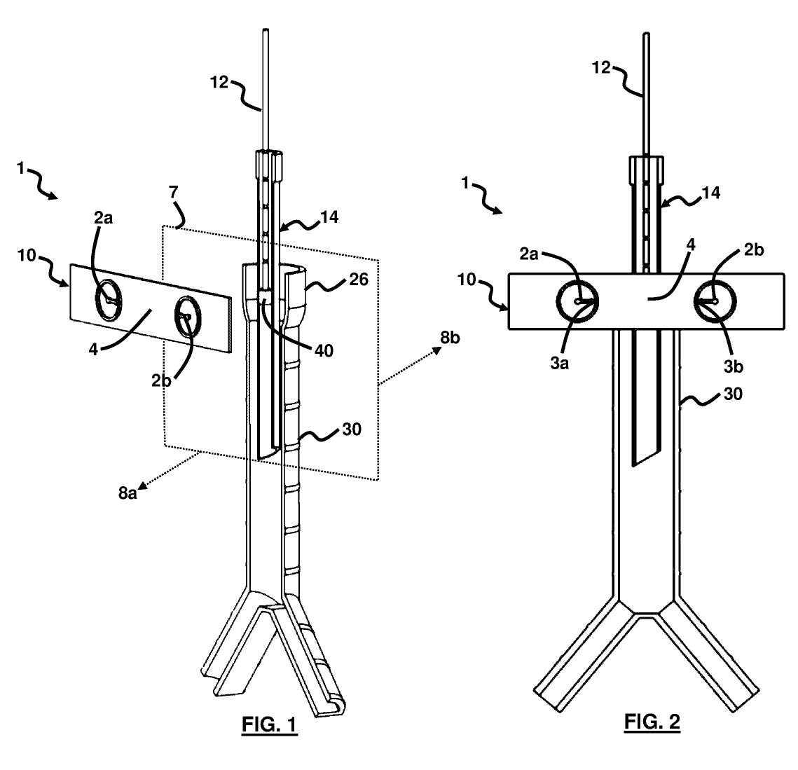

As mentioned, there is a need to develop a new and improved technique for proper ET tube placement in a patient. The embodiments herein provide an ET tube probe that uses a magnet system to allow for proper ET tube placement. Referring now to the drawings, and more particularly to FIGS. 1 through 11, where similar reference characters denote corresponding features consistently throughout the figures, there are shown preferred embodiments.

FIGS. 1 through 3 illustrate a probe system 1 configured to identify a proper placement of a tube (e.g., an ET tube 14, in one embodiment) that is placed inside a human body (e.g., in the larynx 26 and trachea 30, in one embodiment). The system 1 comprises a longitudinal member 12 and a mechanism 10 comprising at least one metallic needle (e.g., in one embodiment, a pair of needles 2a, 2b each comprising magnetically induced metallic points 3a, 3b are provided). The longitudinal member 12 comprises a magnet 40. The longitudinal member 12 is configured to be inserted into an ET tube 14 that is configured to be inserted in the human body (e.g., larynx 26 and down to the trachea 30, in one embodiment). Once the longitudinal member 12 and magnet 40 are positioned in the ET tube 14, the mechanism 10 is positioned over the outside of the human body (e.g., the neck area, in one embodiment) over the general proximate location of where the magnet 40 is located on the corresponding inside of the human body and the combination of the longitudinal member 12 and the ET tube 14 are moved until the pair of needles 2a, 2b align with one another, which indicates that the pair of needles 2a, 2b are aligned with the magnet 40. Plane 7 in FIG. 1 depicts the general indication of the inside/outside dividing plane of the neck area of the human body such that direction 8a indicates the direction towards the outside of the human body and direction 8b indicates the direction towards the inside of the human body, and accordingly in one embodiment plane 7 represents human skin or hair, etc. This indicates that the ET tube 14 has been properly placed inside the human body, and once this is confirmed by an X-ray the longitudinal member 12 and magnet 40 are removed from the ET tube 14. The magnet 40 comprises a magnetic strength that is sufficient to allow its magnetic qualities to be detected by the pair of needles 2a, 2b from outside the human body (e.g., the neck area, in one embodiment), while the magnet 40 is inserted inside the human body (e.g., larynx 26, in one embodiment).

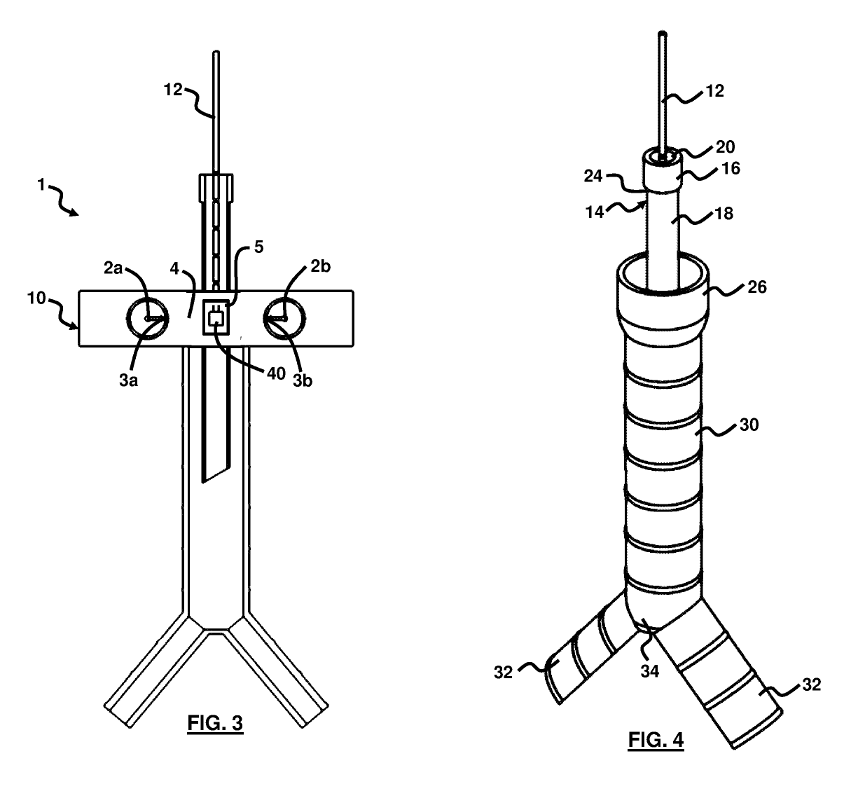

FIGS. 2 and 3 illustrate how the mechanism 10 is placed over the proximate location of the magnet 40. In one embodiment, the mechanism 10 may be configured as a plastic, single-piece board with the pair of needles 2a, 2b configured thereon or embedded therein with a space 4 that separates the pair of needles 2a, 2b from one another. This space 4 may be configured as a planar portion of the mechanism 10 as provided in FIGS. 1 and 2, or the space 4 may be configured with a hole 5 as provided in FIG. 3.

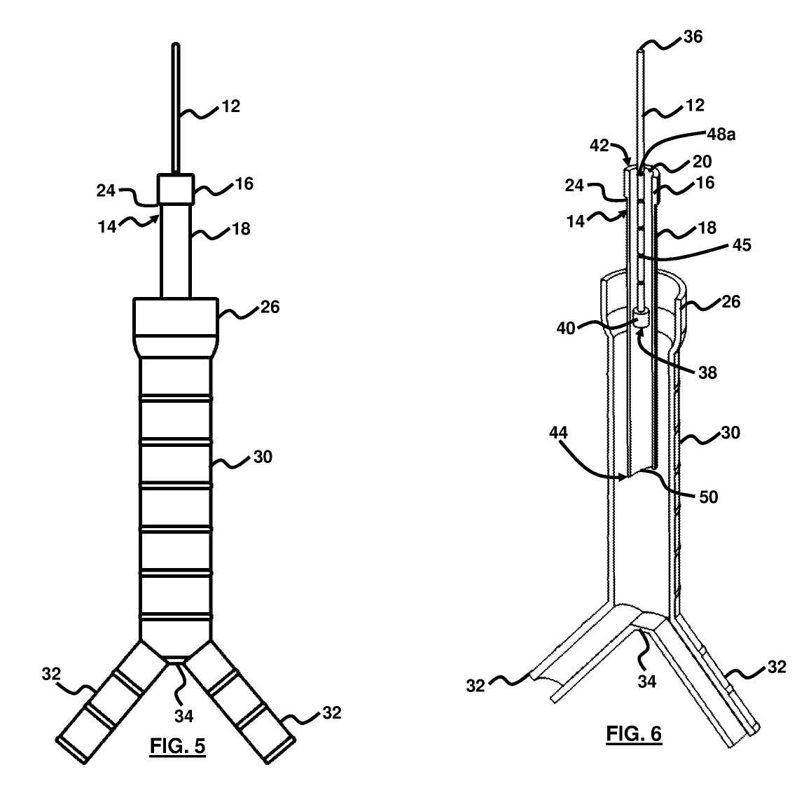

FIGS. 4 and 5, with respect to FIGS. 1 through 3, illustrate the longitudinal member 12 inserted into the ET tube 14 inserted into the larynx 26 and trachea 30. The trachea 30 terminates at the carina 34 before splitting to the bronchi 32. The ET tube 14 may be conventionally configured and comprises a hollow upper portion 16 that leads to an elongated portion 18. A lip 24 is configured between the confluence of the upper portion 16 and elongated portion 18. The upper portion comprises an opening 20 to permit the longitudinal member 12 to be inserted and retracted in/from the ET tube 14.

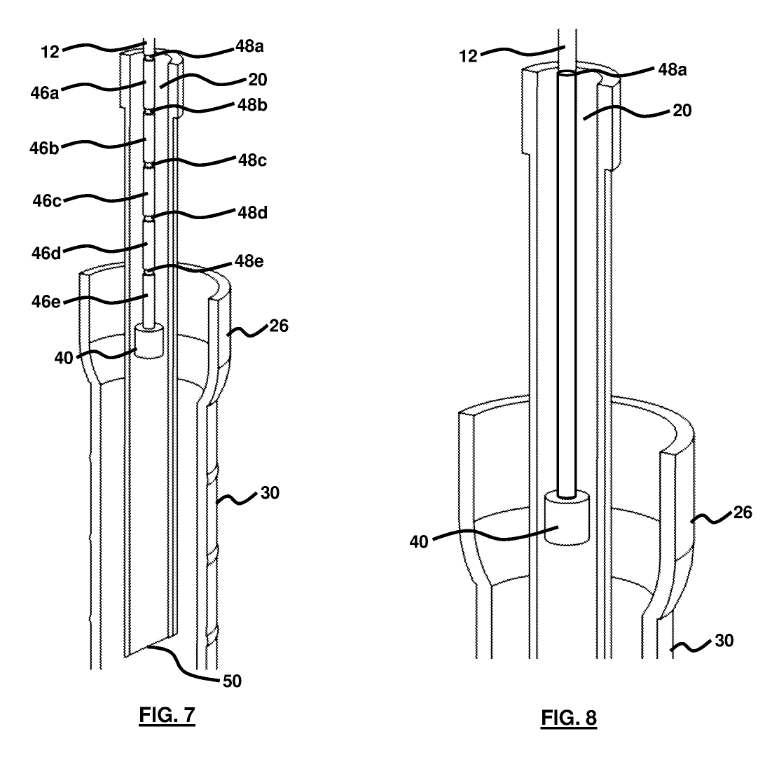

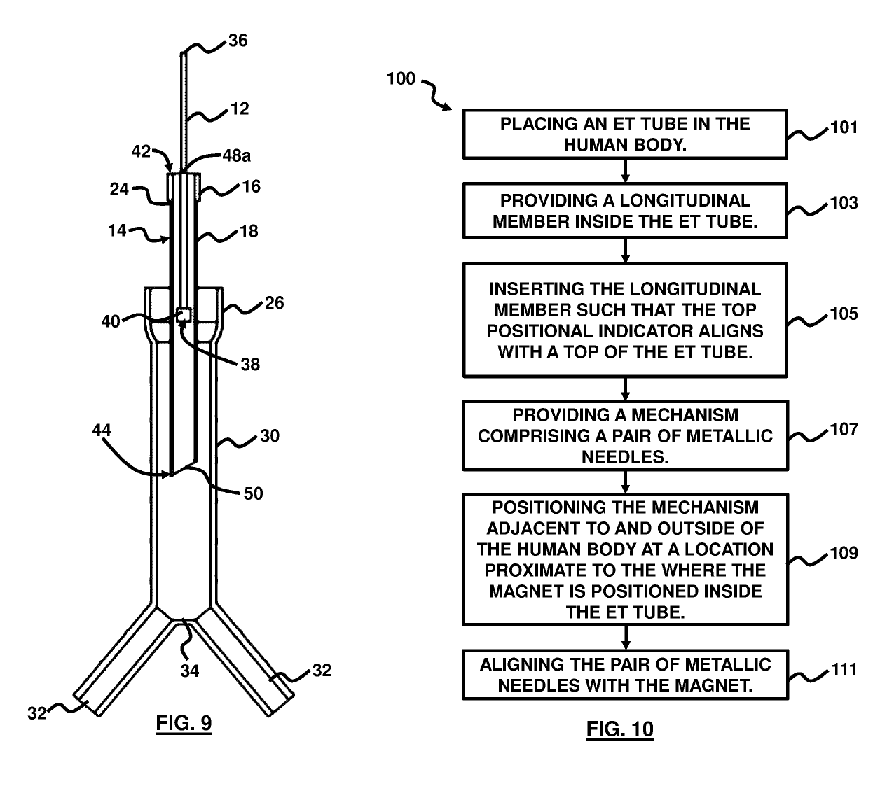

FIGS. 6 through 9, with reference to FIGS. 1 through 5, illustrate various cross-sectional views and embodiments of the ET tube 14 inside the human body (e.g., larynx 26, trachea 30, carina 34, and bronchi 32) with the longitudinal member 12 inserted therein. The ET tube 14 is shown with the open top end 42 and an open bottom end 44 comprising an angled tip 50. The longitudinal member 12 comprises a first end 36 and an oppositely positioned second end 38. The magnet 40 is positioned at the second end 38 of the longitudinal member 12. At least one positional indicator 48a-48e is arranged on the longitudinal member 12. The longitudinal member 12 is configured to be inserted into the ET tube 14 that is placed in the human body (e.g., larynx 26 and trachea 30), wherein the at least one metallic needle 2a, 2b of mechanism 10 (of FIGS. 1 through 3) is configured to align with the magnet 40 when the mechanism 10 is placed adjacent to and outside of the human body. The longitudinal member 12 may comprise plastic, in one embodiment. The at least one positional indicator 48a-48e may be embodied as markings 45 on the longitudinal member 12 with a top positional indicator 48a, in one embodiment as shown in FIG. 6. In the embodiment shown in FIG. 7, the longitudinal member 12 may comprise areas 46a-46e comprising a first width, and the at least one positional indicator 48a-48e comprising a second width. The first width may be different than the second width, in one embodiment. The first width may be greater than the second width, in one embodiment. In the embodiment shown in FIGS. 8 and 9, the longitudinal member 12 comprises only one top positional indicator 48a, which may be embodied as a marking or a depressed area having a width smaller than the width of the other areas of the longitudinal member 12.

The magnet 40 may be thicker than the longitudinal member 12, in one embodiment. The top positional indicator 48a may be adapted to align with a top 42 of the ET tube 14 to indicate a proper placement of the longitudinal member 12 relative to the ET tube 14, as shown in FIGS. 6 through 9. In an exemplary embodiment, the distance between the second end 38 and the top at least one positional indicator 48a is approximately 25 cm. The longitudinal member 12 is freely movable relative to the ET tube 14. The longitudinal member 12 is removable from the ET tube 14. The pair of metallic needles 2a, 2b is configured to become horizontally positioned with respect to one another upon alignment with the magnet 40. The space 4 (with or without a hole 5) is configured to be positioned over the magnet 40 upon proper placement of the ET tube 14 in the human body (e.g. larynx 26 and trachea 30), wherein the at least one metallic needle (e.g., pair of needles 2a, 2b) aligns with the magnet 40 upon the proper placement.

The larynx 26 provides for a better placement marker than lips (as in the conventional medical practice) because from the larynx 26 to the middle of the trachea 30 is only approximately 5 cm, with no anatomical variations therebetween. In use, after the ET tube 14 is introduced into the trachea 30, the longitudinal member 12 is introduced into the ET tube 14 until the second end 38 of the longitudinal member 12 reaches the level of the larynx 26. Then, the upper end 42 of the ET tube 14 is brought to the position of the top positional indicator 48a on the longitudinal member 12 resulting in a perfect location of the ET tube tip 50 to be approximately 5 cm away from the larynx 26, and in the middle of the trachea 30 (e.g., trachea is approximately 10 cm in length). The location and positioning of the mechanism 10 with respect to the larynx 26 is configured to allow the larynx 26 to be positioned between the pair of needles 2a, 2b such that when the mechanism 10 is positioned over the neck area, for example, over the general area where the larynx 26 is located, the pair of needles 2a, 2b begin to point towards the magnet 40 such that the points 3a, 3b of the pair of needles 2a, 2b are configured to be magnetically induced to allow the pair of needles 2a, 2b to rotate until they point towards the underlying magnet 40. The pair of needles 2a, 2b continue to rotate until they line up in a substantially horizontal position with each needle 2a, 2b pointing towards the magnet 40, and thus the pair of needles 2a, 2b essentially point towards each other. In practice, the mechanism 10 may be positioned over the patient's neck before the ET tube 14 and longitudinal member 12 are inserted down the patient's throat such that the pair of needles 2a, 2b first begin to point upwards towards the patient's head since the magnet 40 is still not in the larynx 26 or trachea 30, and as the ET tube 14 with longitudinal member 12 comprising the magnet 40 begins to travel down the patient's throat towards the trachea 30, the pair of needles 2a, 2b begin to rotate until they line up substantially horizontally with respect to each other and with respect to the underlying magnet 40.

In another embodiment, the ET tube 14 and longitudinal member 12 are inserted in the patient's throat first to a general estimate of where the patient's larynx 26 is located and then the mechanism 10 is positioned over the neck area to further fine tune the proper placement of the ET tube 14. The process is adjusted and repeated until the upper end 42 of the ET tube 14 is brought to the position of the top positional indicator 48a on the longitudinal member 12 thereby indicating proper placement of the ET tube 14 inside the patient. An X-ray may be used to confirm the proper placement of the ET tube 14, however the embodiments herein provide a technique to eliminate the guesswork involved in determining whether an ET tube 14 has been properly placed inside a patient, and also eliminates the need for multiple time-consuming and costly X-rays in order to confirm this proper placement.

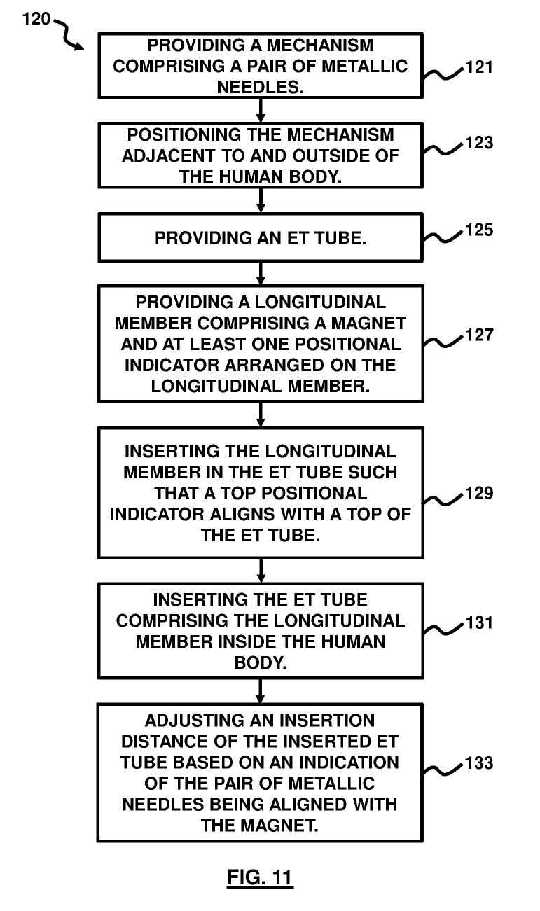

FIGS. 10 and 11, with reference to FIGS. 1 through 9, illustrate flow diagrams according to the embodiments herein. As shown in FIG. 10, a method 100 of identifying the proper placement of an ET tube 14 that is placed inside a human body (e.g., larynx 26 and trachea 30) is provided according to a first embodiment. The method comprises placing (101) an ET tube 14 in the human body (e.g., larynx 26 and trachea 30); providing (103) a longitudinal member 12 inside the ET tube 14, wherein the longitudinal member 12 comprises a first end 36, an oppositely positioned second end 38, a magnet 40 positioned at the second end 38, and at least one positional indicator 48a-48e arranged on the longitudinal member 12; inserting (105) the longitudinal member 12 such that a top positional indicator 48a aligns with a top 42 of the ET tube 14; providing (107) a mechanism 10 comprising a pair of metallic needles 2a, 2b; positioning (109) the mechanism 10 adjacent to and outside of the human body at a location proximate to the where the magnet 40 is positioned inside the ET tube; and aligning (111) the pair of metallic needles 2a, 2b with the magnet 40. The method may further comprise moving both the longitudinal member 12 and the ET tube 14 until the pair of metallic needles 2a, 2b are horizontally positioned with respect to one another. The moving of both the longitudinal member 12 and the ET tube 14 until the pair of metallic needles 2a, 2b is horizontally positioned with respect to one another occurs simultaneously such that the longitudinal member 12 and the ET tube 14 move in unison, in one embodiment. A top positional indicator 48a is adapted to align with a top 42 of the ET tube 14 to indicate a proper placement of the longitudinal member 12 relative to the ET tube 14. The longitudinal member 12 is freely movable relative to the ET tube 14. Moreover, the longitudinal member 12 is removable from the ET tube 14.

As shown in FIG. 11, a method 120 of identifying a proper placement of an ET tube 14 that is placed inside a human body (e.g., larynx 26 and trachea 30) is provided according to a second embodiment. The method comprises providing (121) a mechanism 10 comprising a pair of metallic needles 2a, 2b; positioning (123) the mechanism 10 adjacent to and outside of the human body; providing (125) an ET tube 14; providing (127) a longitudinal member 12 comprising a magnet 40 and at least one positional indicator 48a-48e arranged on the longitudinal member 12; inserting (129) the longitudinal member 12 in the ET tube 14 such that a top positional indicator 48a aligns with a top 42 of the ET tube 14; inserting (131) the ET tube 14 comprising the longitudinal member 12 inside the human body (e.g., larynx 26 and trachea 30); and adjusting (133) an insertion distance of the inserted ET tube 14 based on an indication of the pair of metallic needles 2a, 2b being aligned with the magnet 40.

In order to provide an accurate location of the tip 50 of the ET tube 14 in the trachea 30, after placement of the ET tube 14, if the post placement X-ray shows an incorrect position of the tip 50 of the ET tube (e.g., too high or low in the trachea 30), then the probe system 1 will correct the situation allowing the ET tube 14 to be pulled up or pushed down in the trachea 30 without having to repeat a confirmatory X-ray.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An endotracheal (ET) tube probe system configured to identify a proper placement of an ET tube that is placed inside a human body, said system comprising:
a longitudinal member comprising a first end and an oppositely positioned second end;
a magnet positioned at said second end of said longitudinal member;
at least one positional indicator arranged on said longitudinal member; and
a mechanism comprising at least one metallic needle,
wherein said longitudinal member is configured to be inserted into said ET tube that is placed in said human body, and
wherein said at least one metallic needle is configured to align with said magnet when said mechanism is placed adjacent to and outside of said human body.

2. The system of claim 1, wherein said longitudinal member comprises plastic.

3. The system of claim 1, wherein said at least one positional indicator comprise markings on said longitudinal member.

4. The system of claim 1, wherein said longitudinal member comprises a first width, and wherein said at least one positional indicator comprises a second width.

5. The system of claim 4, wherein said first width is different than said second width.

6. The system of claim 1, wherein said magnet is thicker than said longitudinal member.

7. The system of claim 1, wherein a top of said at least one positional indicator is adapted to align with a top of said ET tube to indicate a proper placement of said longitudinal member relative to said ET tube.

8. The system of claim 1, wherein said longitudinal member is freely movable relative to said ET tube.

9. The system of claim 1, wherein said longitudinal member is removable from said ET tube.

10. The system of claim 1, wherein said at least one metallic needle comprises a pair of metallic needles.

11. The system of claim 10, wherein said pair of metallic needles is configured to become horizontally positioned with respect to one another upon alignment with said magnet.

12. The system of claim 10, wherein said mechanism comprises a space that separates said pair of metallic needles from each other.

13. The system of claim 12, wherein said space is configured to be positioned over said magnet upon proper placement of said ET tube in said human body, and wherein said at least one metallic needle aligns with said magnet upon said proper placement.

14. A method of identifying a proper placement of an endotracheal (ET) tube that is placed inside a human body, said method comprising:
placing an ET tube in said human body;
providing a longitudinal member inside said ET tube, wherein said longitudinal member comprises a first end, an oppositely positioned second end, a magnet positioned at said second end, and at least one positional indicator arranged on said longitudinal member;
inserting said longitudinal member such that a top of said at least one positional indicator aligns with a top of said ET tube;
providing a mechanism comprising a pair of metallic needles;
positioning said mechanism adjacent to and outside of said human body at a location proximate to said where said magnet is positioned inside said ET tube; and
aligning said pair of metallic needles with said magnet.

15. The method of claim 14, further comprising moving both said longitudinal member and said ET tube until said pair of metallic needles are horizontally positioned with respect to one another.

16. The method of claim 15, wherein the moving of both said longitudinal member and said ET tube until said pair of metallic needles is horizontally positioned with respect to one another occurs simultaneously such that said longitudinal member and said ET tube move in unison.

17. The method of claim 14, wherein a top of said at least one positional indicator is adapted to align with a top of said ET tube to indicate a proper placement of said longitudinal member relative to said ET tube.

18. The method of claim 14, wherein said longitudinal member is freely movable relative to said ET tube.

19. The method of claim 14, wherein said longitudinal member is removable from said ET tube.

20. A method of identifying a proper placement of an endotracheal (ET) tube that is placed inside a human body, said method comprising:
providing a mechanism comprising a pair of metallic needles;
positioning said mechanism adjacent to and outside of said human body;
providing an ET tube;
providing a longitudinal member comprising a magnet and at least one positional indicator arranged on said longitudinal member;
inserting said longitudinal member in said ET tube such that a top of said at least one positional indicator aligns with a top of said ET tube;
inserting said ET tube comprising said longitudinal member inside said human body; and
adjusting an insertion distance of the inserted ET tube based on an indication of said pair of metallic needles being aligned with said magnet.
